(19) United States
(12) Patent Application Publication
(10) Pub. No.: US 2004/0260208 A1
(43) Pub. Date: Dec. 23, 2004

(54) Knee laxity measurement

(76) Inventors: Robert Laprade, Chanhassen, MN (US); Fred Wentorf, Roseville, MN (US); David Strothman, Minnetonka, MN (US); Joseph Dvorak, Minneapolis, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/601,003

(22) Filed: Jun. 20, 2003

(51) Int. Cl. A61B 5/11

(52) U.S. Cl. 600/595

(57) ABSTRACT

A device for measuring a varus rotation angle in a knee comprises a femoral reference member configured to be positioned medially of the knee, a first arm hingedly engaged to the femoral reference member, and a first potentiometer to output an angular relationship between the femoral reference member and the first arm. The device also includes a second arm hingedly engaged to the first arm, and a second potentiometer to output an angular relationship between the first and second arms, wherein the varus rotation angle is determinable from the outputs of the first and second potentiometers. Furthermore, a joint-space opening of the knee joint may be determined using the outputs of the first and second potentiometers.

### TECHNICAL FIELD

This invention relates to measuring joint laxity, and certain embodiments relate to measuring varus rotation in a knee joint.

### BACKGROUND

The human knee joint is a complex structure that provides movement beyond a simple “hinge” joint. Several ligaments, working in combination, provide support for the knee joint in the medial-lateral, anterior-posterior, and axial directions. In response to certain forces, the lower portion of the leg may move with up to six degrees of freedom in reference to the upper portion of the leg. The complexity of the knee structure complicates the measurement and diagnosis of joint laxity (e.g., bone movement in a joint within the constraints of the joint ligaments), especially in the case of knee injuries where ligaments are torn or otherwise damaged. For example, many sports-related knee injuries involve a torn anterior cruciate ligament (ACL) or posterior cruciate ligament (PCL), which is commonly diagnosed by way of magnetic resonance imaging and an evaluation of the anterior-posterior translation of the tibia with respect to the femur. According to some, 10-60% of ACL and PCL injuries are associated with secondary injury to the posterolateral corner of the knee and resultant posterolateral instability. The posterolateral instability, however, is often misdiagnosed or never fully appreciated when concurrent ACL or PCL injuries also exist.

Even if the ACL and PCL injuries are properly diagnosed and treated, a failure to properly treat the posterolateral instability may, over a period of time, lead to increased articular contact pressures, early development of osteoarthritis, meniscus tears, and ACL or PCL graft failures. As is known in the art, varus rotation is one indicator of knee laxity and posterolateral instability. Consequently, medical practitioners have determined that it is desirable to measure the varus rotation angle when diagnosing and treating knee injuries, but a method or device for measuring the varus rotation angle that is both clinically usable and sufficiently accurate does not exist.

A conventional examination of knee laxity involves a medical practitioner using his or her hands to manipulate the tibial portion of the leg with respect to the femoral portion of the leg. This type of examination may include the medical practitioner applying a medial-lateral torque about the knee joint to roughly evaluate the amount of varus rotation. Such examinations are subjective in nature (e.g., personal approximation of the knee joint laxity) and reluctantly depend upon the inherent variability between medical practitioners (e.g., different practitioners apply different loads to the knee joint during examination). Furthermore, when multiple portions of the knee are damaged, such as a torn ACL in combination with damage to the posterolateral corner, these conventional examinations may fail to appreciate the damage to the posterolateral corner and the ensuing posterolateral instability.

In an effort to provide more objective measurements of knee laxity, some apparatuses have been developed to mechanically or electronically measure the tibial displacement and rotation in the knee joint. Such apparatuses include cumbersome devices that are attached to both the femoral and tibial portions of the leg, and frequently, these devices are permanently attached to a chair or tabletop where the patient remains while the knee laxity examination is conducted. The bulky size of these apparatus limits their use in medical clinics.

Another type of device to measure knee laxity is more portable and assists in the diagnosis of ACL and PCL injuries. In particular, this type of device measures the anterior-posterior translation of the tibia with respect to the femur when a force is applied in the posterior direction. While this type of device may permit satisfactory diagnosis of ACL and PCL injuries, it does not provide a sufficiently accurate measurement of the varus angle. Consequently, use of this device may permit a medical practitioner to diagnose and treat an ACL or PCL injury while not fully appreciating the posterolateral instability of the damaged knee, which as previously described, may lead to misdiagnosis and further knee damage.

### SUMMARY

In accordance with one embodiment of the invention, a device for measuring a varus rotation angle in a knee of a leg includes a femoral reference member positionable in a reference position against the leg on a medial side of the knee, and a first arm hingedly engaged to the femoral reference member. The first arm extends from the femoral reference member toward a tibial portion of the leg when the femoral reference member is positioned in the reference position. A second arm is hingedly engaged to the first arm and extends from the first arm away from the knee when the femoral reference member is positioned in the reference position. The second member is fastenable to the leg so that the second arm remains in a substantially fixed relationship with the tibial portion of the leg. Furthermore, the device includes a first measuring device that measures a first displacement angle of the first arm in relation to the femoral reference member, and a second measurement device that measures a second displacement angle of the second arm in relation to the first arm. The varus rotation angle is determinable from the first and second displacement angles.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a measuring device in accordance with one embodiment of the invention.

FIG. 2 is a diagram of a system including the device of FIG. 1, shown in a side view and secured to the medial side of a human leg.

FIG. 3 is a frontal view of a human leg and illustrates the forces applied to the leg by the device shown in FIGS. 1-2.

FIGS. 4A-B are frontal views of portions of a femur and a tibia in a knee joint.

FIGS. 5A-B are frontal views of portions of a femur and a tibia in a knee joint, illustrating various displacements of the tibia with respect to the femur.

Like reference symbols in the various drawings indicate like elements. Drawings are not necessarily to scale.

### DETAILED DESCRIPTION OF ILLUSTRATIVE EMBODIMENTS

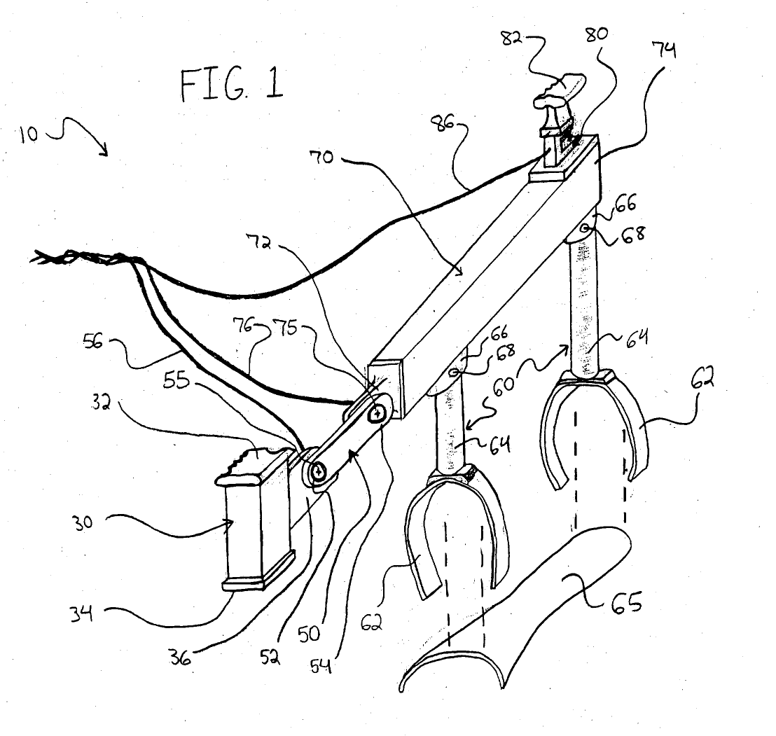

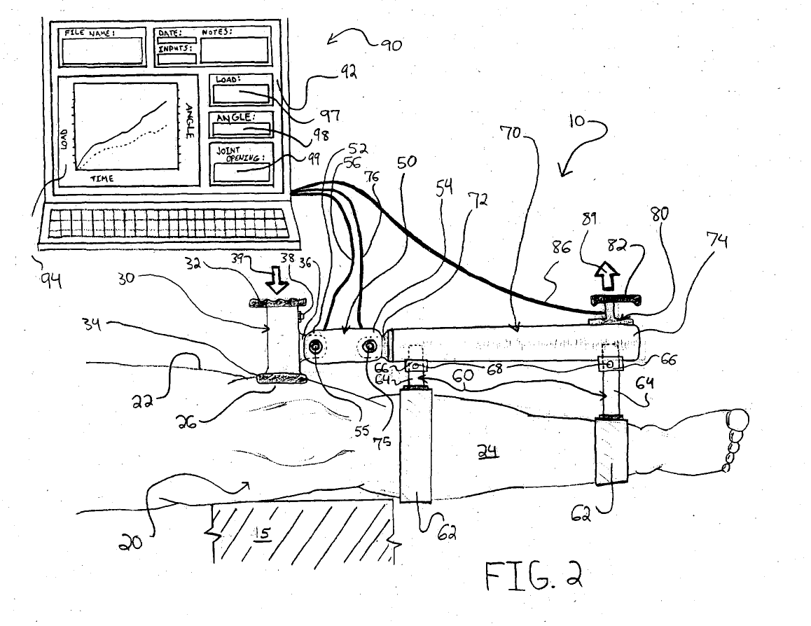

FIGS. 1 and 2 show a knee laxity measuring device 10 in accordance with one embodiment of the invention The device 10 has a femoral reference member 30 that is configured to rest on the medial side of the knee, and a first reference arm 50 that is hingedly engaged to the femoral reference member 30. A first measuring device 55 measures the angular displacement of the first reference arm 50 with respect to the femoral reference member 30 and outputs a signal to a computing device 90. A second reference arm 70 is hingedly engaged to the first reference arm 50, thus, forming a device having multiple degrees of freedom. The second reference arm 70 may be fastened to the tibial portion 24 of the leg 20 to maintain the second arm 70 in a substantially fixed relationship to the tibial portion. A second measuring device 75 measures the angular displacement of the second arm 70 with respect to the first arm 50 and outputs a signal to the computing device 90. The varus rotation angle may be accurately determined from the angular displacement measurements from the first and second measuring devices 55 and 75.

Briefly, the general operation of the device involves positioning the femoral reference member 40 over the medial side of the knee joint and attaching the second reference arm 70 to the tibial portion 24 of the leg 20. To determine the varus rotation angle and the joint-space opening, a medical practitioner would press the femoral reference member 30 downward (e.g., in the lateral direction) on the medial side of the knee joint such that the femur and knee joint are substantially restrained between the femoral reference member 30 and a surface 15. The medical practitioner would pull upward (e.g., in the medial direction) on a handle potion 82 connected to the second reference arm 70 so as to create a torque on the knee joint in the medial-lateral plane. Under this loaded condition, the tibia may experience a medial-lateral translation and a varus rotation, and the first and second reference arms 50 and 70 account for such tibial movement by undergoing angular displacements. These angular displacements are measured by the first and second measurement devices 55 and 75, and the computing device 90 uses the measurements to accurately determine the varus rotation angle while accounting for any change in center of rotation of the tibia caused by the medial-lateral translation (though not necessarily calculating a value for the medial-lateral translation). The device 10 is a suitable size for regular use in a medical clinic and may be easily transported from room to room in a clinic. Preferably, the length of the device is less than 24 inches, and certain embodiments are less than 18 inches in length.

Looking in more detail to the components of the device 10 shown in FIGS. 1 and 2, the femoral reference member 30 may include a handle portion 32 to be grasped by a medical practitioner when the device 10 is operated (explained in more detail below). A start button 38 (FIG. 2) may be positioned optionally on the femoral reference member 30 such that the start button 38 is accessible while handle portion 36 is being grasped. A contact pad 34 may be affixed to the femoral reference member 30 so as to provide a surface that contacts a medial side 22 of a patient's leg 20 when the device 10 is operated. In this embodiment, the handle portion 32 and contact pad 34 are positioned on opposing sides of the femoral reference member 30.

The first reference arm 50 is engaged with the femoral reference member 30 such that the first reference arm 50 may move with one degree of freedom. In this embodiment, a first end 52 of the first reference arm 50 is hingedly engaged with a linking portion 36 of the femoral reference member 30 so that the first reference arm 50 may rotate with respect to the femoral reference member 30. A first measuring device 55, such as a transducer or potentiometer, may be mounted to the knee laxity measuring device 10 so as to measure the movement of the first reference arm 50 with respect to the femoral reference member 30. In the embodiment shown in FIGS. 1 and 2, the first measuring device 55 is a potentiometer that is capable of determining the angular displacement of the first reference arm 30 with respect to the femoral reference member 30. The potentiometer 55 may be mounted near the first end 52 of the first reference arm 50 such that the potentiometer 55 is positioned at the interface of the first end 52 and the linking portion 36. Any signals indicative of measurement from the potentiometer 55 may be output through a connecting wire 56 or other transmission medium to a computing device 90 (FIG. 2) for data computation or storage (described in more detail below).

The second reference arm 70 is engaged with a second end 54 of the first reference arm 50 such that the second reference arm 70 may move with one degree of freedom. In this embodiment, a first end 72 of the second reference arm 70 is hingedly engaged with the second end 54 of the first reference arm so that the second reference arm 70 may rotate with respect to the first reference arm 50. As perhaps best shown in FIG. 1, the rotational movement of the first reference arm 50 is in substantially the same plane or a parallel plane as the rotational movement of the second reference arm 70, but other embodiments are not limited to such a configuration.

A second measuring device 75 may be mounted to the knee laxity measuring device 10 so as to measure the movement of the second reference arm 50 with respect to the first reference arm 50. In the embodiment shown in FIGS. 1 and 2, the second measuring device is a potentiometer 75 that is capable of determining the angular displacement of the second reference arm 30 with respect to the first reference arm 50. The potentiometer 75 may be mounted near the first end 72 of the second reference arm 70 such that the potentiometer 75 is positioned at the interface of the first end 72 and the second end 54 of the first reference arm 50. Any signals indicative of measurement from the potentiometer 75 may be output through a connecting wire 76 to the computing device 90 for data computation or storage.

As shown in FIGS. 1-2, a load measuring device 80 is mounted near a second end 74 of the second reference arm 70. The load measuring device 80 is capable of measuring a load, such as a force or a torque, applied to the device 80 near the second end 74. In this embodiment, the load measuring device 80 is a load cell that has a handle portion 82 adapted to be grasped by a medical practitioner when the device 10 is operated (described in more detail below). As such, the load cell 80 is capable of measuring the force between the handle portion 82 and the second reference arm 70. Any signal indicative of the load measurement from the load cell 80 may be output through a connecting wire 86 to the computer device 90.

One or more tibial attachment devices 60 may be mounted to the second reference arm 70. When in operation, the tibial attachment devices 60 are used to maintain the tibial portion 24 (FIG. 2) of the leg 20 in a substantially fixed relationship to the second reference arm 70. In this embodiment, two tibial attachment devices 60 are spaced apart along the tibial portion of the leg, but there may be any number of attachment devices is in other embodiments. The tibial attachment device 60 may include a strap 62 or other similar mechanism for securing the tibial portion 24 of the leg 20 to the tibial attachment device 60. Each strap 62 may use conventional securing means, such as a buckle or VELCRO strips, and may have a thickness or width that is necessary to maintain the tibial portion 24 of the leg 20 in a fixed relationship to the second reference arm 70. The tibial attachment device 60 may be adjustably mounted to the second reference arm 70. In this embodiment, the tibial attachment 60 includes an extension pole 64 that is attached to the strap 62 at one end and mounted through a socket 66 at an opposing end. The socket 66 may include a locking mechanism 68 so as to retain the position of the extension pole 64 after proper adjustment. Optionally, the tibial attachment device 60 may include a partial cast 65 (FIG. 1) that is positioned between the strap 62 and the tibia portion 24 of the leg 20. The partial cast 65 may be made from a moldable material, such as plastic, that is specifically conformed to the shape of a patient's tibial portion 24. According to this embodiment, the partial cast 65 and the straps 62 operate in combination to maintain the tibial portion 24 of the leg 20 in a fixed relationship to the second reference arm 70.

Referring to FIG. 2, the device 10 may be attached to the tibial portion 24 of the leg 20 using the tibial attachment devices 60 so that the device 10 is positioned medially of the leg 20. The contact pad 34 of the femoral reference member 30 may rest on the medial femoral epicondyle 26 of the leg 20 when the device 10 is properly attached to the tibial portion 24. The positioning of the device in this manner permits a load substantially in the medial-lateral plane to be applied to the leg 20, for example, by pulling on the handle portion 82 with a force 89.

Looking in more detail to the operation of the device 10, a medical practitioner may properly secure the device 10 to the medial side of the leg 20 while the lateral side of the leg 20 (or at least a portion thereof) rests on a surface 15. When the leg 20 and device 10 are properly positioned, the practitioner may reset or “zero” the measuring devices 55 and 75 and the load measure device 80. While FIG. 2 shows the first and second reference arms 50 and 70 in substantially parallel positions, the device 10 may be “zeroed” (and subsequently operated) when the first and second reference arms 50 and 70 are in nonparallel positions. When the practitioner is ready to apply a load to the leg 20 using the device 10, the practitioner may press the start button 38, which may commence recording of measurement data from the measurement devices 55 and 75 and the load measuring device 80.

As shown in the embodiment of FIG. 2, the practitioner may apply a load substantially in the medial-lateral plane by applying a force 39 in the lateral direction (downward direction as shown in FIG. 2) to the handle portion 32 of the femoral reference member 30 and applying a force 89 in the medial direction (upward direction as shown in FIG. 2) to the handle portion 82 of the load cell 80. By applying the force 39 to the femoral reference member 30, the motion of the upper leg and knee joint, especially the femoral condyles, is substantially restrained against the surface 15, but the force 89 may cause displacement of the tibia with respect to the femur (described in more detail below). Any displacement caused by the forces 39 and 89 may be detected by the potentiometers 55 and 75 such that the first potentiometer 55 measures the angular displacement of the first reference arm 50 with respect to the femoral reference member 30 and the second potentiometer 75 measures the angular displacement of the second reference arm 70 with respect to the first reference arm 50. After the practitioner has completed the application of the load for a certain amount of time or when a certain threshold has been satisfied, the practitioner may end the device operation by pressing the start button 38 or otherwise ceasing the data output from the device 10.

While the load is being applied to the leg 20 and the device 10 is in operation, the potentiometers 55 and 75 and the load cell 80 may transmit signals indicative of their respective measurements to the computing device 90 via the connecting wires 56, 76, and 86. The computing device 90 may be electrically connected to a display 92 that is capable of displaying data related to the measurements from the potentiometers 55 and 75 and the load cell 80. In one embodiment, the computing device 90 may determine a varus rotation angle (described in more detail below) using the angular measurements from the potentiometers 55 and 75, and the display 92 may include a real-time portion 94 that shows the varus rotation angle, the joint-space opening, or other measurements in real-time. For example, the practitioner may view a portion 94 of the display 92 that shows a plot of varus rotation angle versus time. In addition, the portion 94 may show the load that is being applied by the practitioner in real-time while the device 10 is being operated. The display 92 may show other data determined by the computing device 90, such as varus-rotation-angle data 98 at a particular load or joint-space-opening data 99 (described in more detail below) at a particular load. Furthermore, the computing device 90 and display 92 may enable a medical practitioner to enter certain inputs, such as a patient's weight and certain dimensions, which may subsequently be used by the computing device 90 when determining the applied torque load, the joint-space opening, or other measurements.

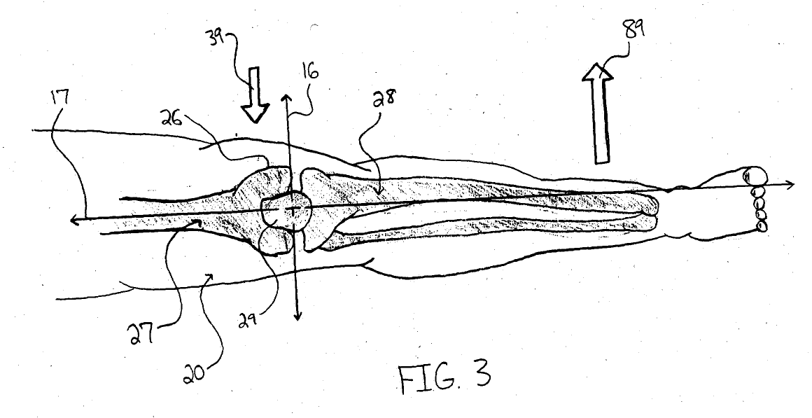

FIG. 3 shows a bone structure inside leg 20, which includes a femur 27, patella 29, and a tibia 28, and a fibula. The medial-lateral plane in the leg 20 is defined by a medial-lateral axis 16 and a femoral axis 17, which are substantially perpendicular to each other. When the device 10 (not shown in FIG. 3) is in operation, and the forces 39 and 89 are applied to create a torque substantially in the medial-lateral plane, the displacement of the tibia 28 with respect to the femur 27 is not necessarily a simple “hinged” movement in the medial-lateral plane.

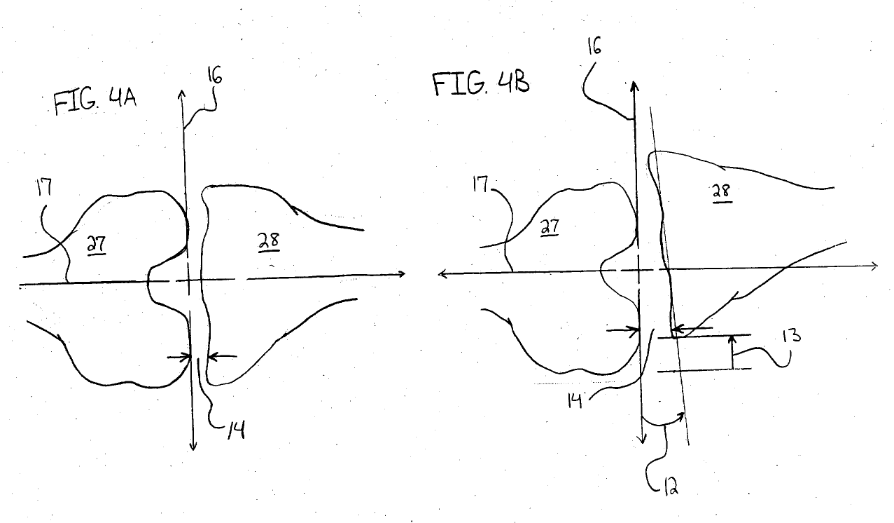

As shown in FIGS. 4A-4B, the displacement of the tibia 28 with respect to the femur 27 under the previously described load conditions may involve both a medial-lateral translation 13 and a varus rotation 12. In one example, FIG. 4A shows a femur 27 and a tibia 28 under unloaded conditions such that the varus rotation angle is negligible and a joint-space opening 14 is relatively normal. FIG. 4B shows the femur 27 and tibia 28 when a torque load is applied on the leg 20 by the forces 39 and 89 (FIGS. 2-3). Under this loading, the movement of the femur 28 is substantially restrained while the tibia 28 may be displaced by the medial-lateral translation 13 and the varus rotation 12. The medial-lateral translation 13 and the varus rotation 12 may cause a measurable increase in the joint-space opening 14. The joint space opening 14 may be determined as a function of the varus rotation angle 12 and the width dimension between the patient's lateral tibial plateau and femoral condyle.

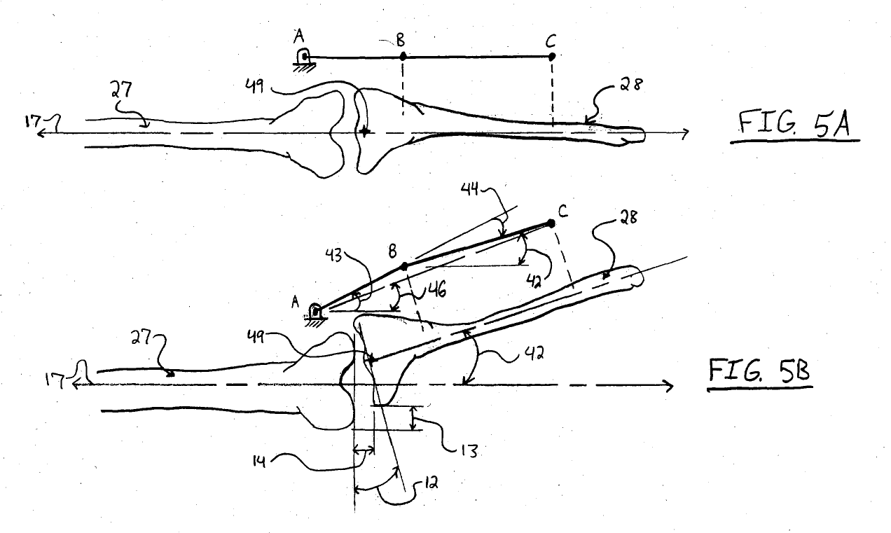

FIGS. 5A-B show a linkage ABC that may represent, for illustrative purposes only, the movement of first and second reference arms 50 and 70 of the measuring device 10 when the tibia 28 is displaced with respect to the femur 27 as previously described in FIG. 4B. FIGS. 5A-B are intended to illustrate the error-causing effects of a device that uses a single potentiometer to measure the varus rotation angle 12 and the increased accuracy of the measuring device 10 shown in FIGS. 1-2 to measurement of the varus rotation angle 12. FIGS. 5A-B do not illustrate limitations upon the operation of the device 10, the construction of the device 10, or the computation of the varus rotation angle 12.

Link AB is a simplified model of the movement of the first reference arm 50, which is not necessarily to scale. Similarly, link BC is a simplified model of the movement of the second reference arm 70, which is not necessarily to scale. In this simplified example, the link AB is hingedly engaged at grounded pin A (e.g., a simplified model of the femoral reference member 30 pressed against the medial femoral epicondyle), and link BC is hingedly engaged with link AB at pin B. Also in this example, link BC is secured to the tibial portion of the leg such that the tibia 28 is maintained in a fixed relationship to link BC.

In FIG. 5A, the femur 27 and tibia 28 are under unloaded conditions, and the varus rotation angle 12 and the medial-lateral translation 13 are negligible. At this point, the angular displacement of link AB (with respect to ground pin A) and the angular displacement if link BC (with respect to link AB) may be considered “zero” such that any displacements of link AB and link BC may be measured from the “zeroed” positions.

As shown in FIG. 5B, when a load (forces 39 and 89) from the measuring device 10 is applied to the tibia 28, the tibia 28 undergoes both a medial-lateral translation 13 and a varus rotation 12. As a result of the tibial displacement, link AB undergoes an angular displacement 43 with respect to ground pin A. Because link BC is maintained in a substantially fixed relationship to the tibia 28, link BC also undergoes an angular displacement 44 with respect to link AB. The angular displacement 42 of the tibia 28 in the medial-lateral plane may be accurately approximated from the angular displacements 43 and 44 of the two links using standard linkage analysis. In the example shown in FIG. 5B, angle 42 is equal to the magnitude of angle 43 subtracted by the magnitude of angle 44. The varus rotation angle 12 may be determined using standard geometry analysis. In the illustrative example shown in FIG. 5B, the varus rotation angle 12 is equal to the measured angular displacement 42. When the varus rotation angle 12 is determined, the joint space opening 14 may be computed using simple geometry for a standard-sized knee or based upon measurements obtained from radiographic methods.

FIG. 5B also shows the error-causing effect of using a single reference arm (and a corresponding single potentiometer) to measure the varus rotation angle 12. Single link AC (shown as a dashed line) is hingedly engaged with ground pin A and represents a device using a single reference arm to measure the varus rotation angle. When the tibia 28 is displaced by both a medial-lateral translation 13 and a varus rotation 12, the single link AC undergoes an angular displacement 46. The single link AC does not account for the change in center of rotation 49 caused by the medial-lateral translation 13 of the tibia 28. Consequently, the angular displacement 46 of link AC does not, in general, equal the actual angular displacement 42 of the tibia 28. Unlike the previously described linkage ABC, which uses angular displacements 43 and 44 of two different links to account for the change in center of rotation 49 while measuring the varus rotation 12, the measurement of angle 46 is not sufficiently accurate to determine the varus rotation 12 of the tibia 28.

A number of embodiments of the invention have been described. Nevertheless, it will be understood that various modifications may be made without departing from the spirit and scope of the invention. For example, the first reference arm 50 or the second reference arm 70 may be constructed to extend or contract in length so that the reference arm may be adjusted to fit differing legs sizes. Moreover, the extension poles 64 may be constructed to extend or contract in length so that the attachment straps 62 may be adjusted to different distances from the second reference arm 70 (permitting the attachment device to be adjusted to fit differing leg sizes). Furthermore, the femoral reference member 30, the first and second reference arms 50 and 70, and the extension poles 64 may be made from any substantially rigid material, such as tubular or solid metal material, certain plastic materials, or wood. In addition, the device 10 may be modified to measure the valgus opening of the knee joint using principles similar those described above. Also, the measurements from the device 10 may be recorded using means other than the computing device. Accordingly, other embodiments are within the scope of the following claims.

What is claimed is:

1. A device for measuring a varus rotation angle in a knee of a leg, comprising:
a femoral reference member positionable in a reference position against the leg on a medial side of the knee;
a first arm having a first end hingedly engaged to the femoral reference member and extending from the femoral reference member toward a tibial portion of the leg when the femoral reference member is positioned in the reference position;
a second arm having a first end hingedly engaged to a second end of the first arm and extending from the first arm away from the knee when the femoral reference member is positioned in the reference position, the second member fastenable to the leg so that the second arm remains in a substantially fixed relationship with the tibial portion of the leg;
a first measuring device that measures a first displacement angle of the first arm in relation to the femoral reference member, the first displacement angle measurable when a load is applied to the tibial portion of the leg; and
a second measurement device that measures a second displacement angle of the second arm in relation to the first arm, the second displacement angle measurable when the load is applied to the tibial portion of the leg,
wherein the varus rotation angle is determinable from the first and second displacement angles.

2. The device of claim 1, wherein the varus rotation is determinable from the first and second displacement angles while displacements of the first and second arms account for a medial-lateral translation of a tibia in the knee.

3. The device of claim 1, wherein a joint-space opening is determinable from the first and second displacement angles the first and second measurement devices.

4. The device of claim 1, wherein the combined length of the femoral reference member, the first arm, and the second arm is less than 24 inches.

5. The device of claim 4, wherein the combined length of the femoral reference member, the first arm, and the second arm is less than 18 inches.

6. The device of claim 1, wherein the femoral reference member is positionable in the reference position against the leg on a medial femoral epicondyle of the knee.

7. The device of claim 6, further comprising a contact pad on a femoral reference member positionable on the medial femoral epicondyle.

8. The device of claim 1, wherein the second arm is fastenable to the tibial portion of the leg such that the second arm is positioned medially of the leg and in a substantially fixed relationship to the tibial portion.

9. The device of claim 8, further comprising one or more attachment devices connected to the second arm, each attachment device having a strap operable to fasten the second arm to the tibial portion of the leg.

10. The device of claim 9, further comprising a partial cast positioned between each strap and the tibial portion of the leg to maintain the second reference arm in a substantially fixed relationship to the tibial portion.

11. The device of claim 1, wherein the first and second measurement devices are potentiometers.

12. A system for measuring displacement of a tibia in a knee of a leg, comprising:
a device for measuring a varus rotation angle, comprising:
a femoral reference member positionable in a reference position against the leg on a medial side of the knee;
a first arm hingedly engaged to the femoral reference member and extending from the femoral reference member toward a tibial portion of the leg when the femoral reference member is positioned in the reference position;
a second arm hingedly engaged to the first arm and extending from the first arm away from the knee when the femoral reference member is positioned in the reference position, the second member fastenable to the leg so that the second arm remains in a substantially fixed relationship with the tibial portion of the leg;
a first measuring device that measures a first displacement angle of the first arm in relation to the femoral reference member; and
a second measurement device that measures a second displacement angle of the second arm in relation to the first arm,
wherein the varus rotation angle is determinable from the first and second displacement angles;
a computing device operable to receive outputs of the first and second measuring devices and to compute the varus rotation angle from the first and second displacement angles; and
a display electrically connected to the computing device to show the varus rotational angle in real-time.

13. The system of claim 12, wherein the display is operable to show a real-time graph of the varus rotation angle versus time.

14. The system of claim 12, wherein the computing device operable to compute a joint-space opening value from the first and second displacement angles and the display is operable to show the joint-space opening value.

15. The system of claim 12, wherein the device for measuring varus rotation angle further comprises a load measuring device connected to the second arm such that the load measuring device is operable to output a measurement of load applied to the second arm.

16. The system of claim 15, wherein the display is operable to show a real-time graph of the measurement of load versus time.

17. The system of claim 12, wherein the device for measuring varus rotation angle further comprises a start button near a handle portion on the femoral reference member.

18. The system of claim 17, wherein the computing device is operable to receive signals from the first and second measurement devices when the start button is engaged.

19. A method of measuring displacement of a tibia in a knee of a leg, comprising:
attaching a knee laxity measuring device to a tibial portion of the leg such that the knee laxity measuring device is positioned medially of the knee, wherein the knee laxity measuring device includes:
a femoral reference member positionable in a reference position against the leg on a medial side of the knee;
a first arm hingedly engaged to the femoral reference member and extending from the femoral reference member toward the tibial portion of the leg when the femoral reference member is positioned in the reference position;
a second arm hingedly engaged to the first arm and extending from the first arm away from the knee when the femoral reference member is positioned in the reference position, the second member fastenable to the leg so that the second arm remains in a substantially fixed relationship with the tibial portion of the leg;
a first measuring device that measures a first displacement angle of the first arm in relation to the femoral reference member; and
a second measurement device that measures a second displacement angle of the second arm in relation to the first arm;
applying a force in the medial direction to the second arm while the femoral reference member substantially restrains movement of the knee; and
determining the varus rotation angle from the first and second displacement angles.

20. The method of claim 19, wherein the second arm is operable to be attached to the tibial portion such that the second arm is positioned medially of the leg and in a substantially fixed relationship to the tibial portion.

21. The method of claim 20, wherein the knee laxity measuring device further comprises one or more attachment devices connected to the second arm, each attachment device having a strap operable to fasten the second arm to the tibial portion of a leg.

22. The method of claim 19, further comprising determining the varus rotation angle from the first and second displacement angles while displacements of the first and second arms account for a medial-lateral translation of the tibia in the knee.

23. The method of claim 22, wherein the varus rotation angle is determined from the first and second displacement angles without an error-causing effect from a change in center of rotation the tibia.

24. The method of claim 19, further comprising determining a joint-space opening from the first and second displacement angles.
